(19) United States
(12) Patent Application Publication
(10) Pub. No.: US 2018/0368718 A1
(43) Pub. Date: Dec. 27, 2018

(54) Smart Headphones

(71) Applicant: Jeffrey E. Koziol, Arlington Heights, IL (US)

(72) Inventor: Jeffrey E. Koziol, Arlington Heights, IL (US)

(21) Appl. No.: 16/014,481

(22) Filed: Jun. 21, 2018

Related U.S. Application Data
(60) Provisional application No. 62/522,849, filed on Jun. 21, 2017.

(51) Int. Cl. A61B 5/0478; A61B 5/04; H04R 1/10

(52) U.S. Cl. CPC A61B 5/0478; H04R 2420/07; H04R 1/1016; A61B 5/04004

(57) ABSTRACT

An on-body, particularly in-ear or on-ear, device, such as a headphone or an ear bud includes an electroencephalogram (EEG) electrode for measuring, monitoring and/or recording data indicative of EEG waves, or electrical activity in a brain, of a person wearing the device. The device can be configured to output data indicative of a baseline EEG comprising for example alpha, beta, gamma, and delta waves, which can be recorded and/or output on a display. Color can be designated as corresponding to certain activity of person wearing on-body device and for visual display color-coded EEG wave data could be summed so the screen color can change intensity and/or shade thereof representative information such as stress or relaxation level of personal wearing on-body device.

This application claims priority to prior U.S. Provisional Patent Application No. 62/522,849, filed Jun 21, 2017, the entire content of which is incorporated herein by reference.

### BACKGROUND OF THE DISCLOSURE

### 1. Field of Disclosure

Generally, exemplary embodiments of the present disclosure relate to the field of activity trackers, wireless or wired wearable technology devices for measuring data such as heart rate, blood pressure, temperature, and other personal health data. Exemplary implementations of certain embodiments of the present disclosure provide systems and methods for measuring a person's health data and further provide a novel portable, wearable sensor for monitoring and/or collecting such data.

### 2. Discussion of the Background of the Disclosure

Conventional methods for measuring personal health data include the use of wearable activity trackers such as wrist bands, chest bands, etc. to continuously monitor a person's heartrate during essentially any activity. Other devices include function-specific apparatuses, such as wrist or arm cuffs for measuring blood pressure. On the other hand, hospitals use monitoring stations with electrodes attached to various body parts to measure and monitor patient's vital signs.

All of these conventional device and techniques for measuring and/or monitoring personal health data have various drawbacks. For example, chest bands are an additional function-specific device that a person may need to wear for monitoring heart rate. Wrist bands may provide additional functionality, such as time display, but may none-the-less interfere with other functional or decorative items a person may wish to wear on their wrists. Blood pressure monitors tend to be bulky and may not provide continuous monitoring. Devices employed in hospital settings are not readily wearable for everyday activities.

### SUMMARY OF THE DISCLOSURE

Exemplary embodiments of the present disclosure address at least such drawbacks by providing systems and methods including an in-ear or on-ear device for obtaining, measuring and/or monitoring a person's health data.

An exemplary embodiment of the present disclosure provides an in-ear or on-ear device, such as a headphone or an ear bud, which includes an electroencephalogram (EEG) electrode for measuring, monitoring and/or recording data indicative of EEG waves, or electrical activity in a brain, of a person wearing the device.

According to an exemplary implementation of embodiments of the preset disclosure, an in-ear or on-ear device can be configured to output data indicative of a baseline EEG comprising for example alpha, beta, gamma, and delta waves. Such data can be recorded and, for example color coded, for output on a display. In yet further exemplary implementation, certain color can be designated as corresponding to a certain activity of a person wearing the in-ear or on-ear device, such as for example a relaxation or meditation where certain EEG waves would predominate. For visual display on a screen, color-coded EEG wave data could be summed together for example as green, so the screen would change from a neutral to shades of green with green becoming more intense as relaxation is achieved. In a similar manner, EEG wave data can be color-coded and displayed on the screen such that red color could signal stress.

In yet further exemplary implementations of the embodiments of the present disclosure, an in-ear or on-ear device can be configured with for example three insulated electrodes to be in contact with the upper frontal and posterior areas of the ear to measure electrical activity from different areas of the brain.

Another exemplary embodiment of the present disclosure provides an in-ear or on-ear device, such as a headphone or an ear bud, which includes a temperature sensor and/or a microphone and/or an LED element. In an exemplary implementation, sensors such as a microphone can be configured to sense a person's pulse wave velocity and/or hear rate. In yet further exemplary implementation, LED element can be configured as a light source in conjunction with a photosensitive element for measuring blood oxygen (O₂). Sensor(s) can also be configure to detect respiration, or any other telemetry measurable by a sensor placed in, on, and/or in the proximity of, an ear, for example in conjunction with an audio devise such as a headphone or an ear bud.

In still further exemplary embodiment of the present disclosure, an in-ear or on-ear device, such as a headphone or an ear bud, can selectively comprise any and/or all of the monitoring elements, sensors, and/or capabilities described above.

In yet other exemplary implementation of the embodiments of the present disclosure, additional sensors can be placed in the vicinity of other body parts, such as in a headband and/or a wrist band, to provide additional sensing capability delivering sensed data via wired and/or wireless communication to a processor disposes within an in-ear and/or an on-ear device according to the embodiments of the present disclosure. Furthermore, an in-ear and/or an on-ear device according to the embodiments of the present disclosure can be configure to communicate with conventional and/or other sensing devices, such as wristbands, chest bands, a pulse sensor in watch, and/or an ankle band, to receive additional data for processing, storage and/or output.

In an exemplary implementation of any of the embodiments of the present disclosure, data indicative of any of the monitored parameters such as EEG, temperature, pulse, respiration, O₂, etc. can be transmitted from the device via wired or wireless connection to a data processing and/or display device, including without limitation a portable communication device, such as a smartphone or a tablet, a portable or desktop computer, or any other data storage, processing and/or display device.

In yet another exemplary configuration of any of the embodiments of the present disclosure, an in-ear or on-ear device can receive control data and/or parameters, via wired or wireless communication, from a data processing and/or display device, including without limitation a portable communication device, such as a smartphone or a tablet, a portable or desktop computer, or any other data storage, processing and/or display device. In yet further exemplary implementation, control data and/or parameters can facilitate an in-ear or on-ear device to selectively output any one or more of the monitored data, such as EEG waves, temperature, pulse, hear rate, and/or respiration. In yet further exemplary implementation, controlling of the selective output can include, without limitation: frequency and/or timing of the measurements and/or output of any of the data sensed by any of the sensors configure in/on the in-ear or on-ear device. In still further exemplary implementation, control data and/or parameters can include instructions for turning on and/or off any one of, or combination of, the sensors configure in/on the in-ear or on-ear device.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein

FIGS. 1A and 1B is an illustrative conceptual diagram showing diagrammatic representation of a device according to an exemplary implementation of exemplary embodiments of the present disclosure.

FIG. 1C is an illustrative conceptual diagram showing diagrammatic representation of a device according to an exemplary implementation of exemplary embodiments of the present disclosure with respect to a human ear.

FIGS. 2A and 2B is an illustrative conceptual diagram showing diagrammatic representation of a device according to another exemplary implementation of exemplary embodiments of the present disclosure.

FIG. 2C is an illustrative conceptual diagram showing diagrammatic representation of a device according to another exemplary implementation of exemplary embodiments of the present disclosure with respect to a human ear.

FIG. 3 is a block diagram illustrating wired and/or wireless communication for one or more devices according to exemplary embodiment of the present disclosure.

FIG. 4 is a block diagram illustrating system according to exemplary embodiment of the present disclosure including one or more devices according to exemplary embodiments of the present disclosure.

FIGS. 5A, 5B, 5C, and 5D are diagrammatic illustrations of examples of displays on connected devices according to embodiments of the present disclosure receiving information from sensing devices according to embodiments of the present disclosure.

FIGS. 6A, 6B, and 6C diagrammatically illustrate examples of interactive displays that can be generated on connected devices according to exemplary implementations of exemplary embodiments of the present disclosure.

FIG. 7A is an illustrative conceptual diagram showing diagrammatic representation of a device according to yet another exemplary implementation of exemplary embodiments of the present disclosure.

FIG. 7B is an illustrative conceptual diagram showing diagrammatic representation of a device according to still further exemplary implementation of exemplary embodiments of the present disclosure with respect to a human ear.

FIG. 7C is an illustrative conceptual diagram showing diagrammatic representation of a device according to yet another exemplary implementation of exemplary embodiments of the present disclosure.

FIG. 8A is an illustrative conceptual diagram showing diagrammatic representation of a device according to yet another exemplary implementation of exemplary embodiments of the present disclosure.

FIG. 8B is an illustrative conceptual diagram showing diagrammatic representation of a device according to yet another exemplary implementation of exemplary embodiments of the present disclosure with respect to a human ear.

FIG. 9A is an illustrative conceptual diagram showing diagrammatic representation of a device according to yet another exemplary implementation of exemplary embodiments of the present disclosure.

FIG. 9B is an illustrative conceptual diagram showing diagrammatic representation of a device according to yet another exemplary implementation of exemplary embodiments of the present disclosure with respect to a human ear.

FIGS. 10A and 10B is an illustrative conceptual diagram showing diagrammatic representation of a device according to yet further exemplary implementation of exemplary embodiments of the present disclosure.

FIG. 10C is an illustrative conceptual diagram showing diagrammatic representation of a device according to yet further implementation of exemplary embodiments of the present disclosure with respect to a human ear.

FIGS. 11A is an illustrative conceptual diagram showing diagrammatic representation of a device according to another exemplary embodiment of the present disclosure.

FIGS. 11B is an illustrative conceptual diagram showing diagrammatic representation of a device according to yet another exemplary embodiment of the present disclosure

FIG. 12 is an illustrative conceptual diagram showing diagrammatic representation of an on-body device according to still further exemplary embodiment of the present disclosure.

### DETAILED DESCRIPTION OF EXEMPLARY EMBODIMENTS

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the described disclosure. Also, descriptions of well-known functions and constructions are omitted for clarity and conciseness.

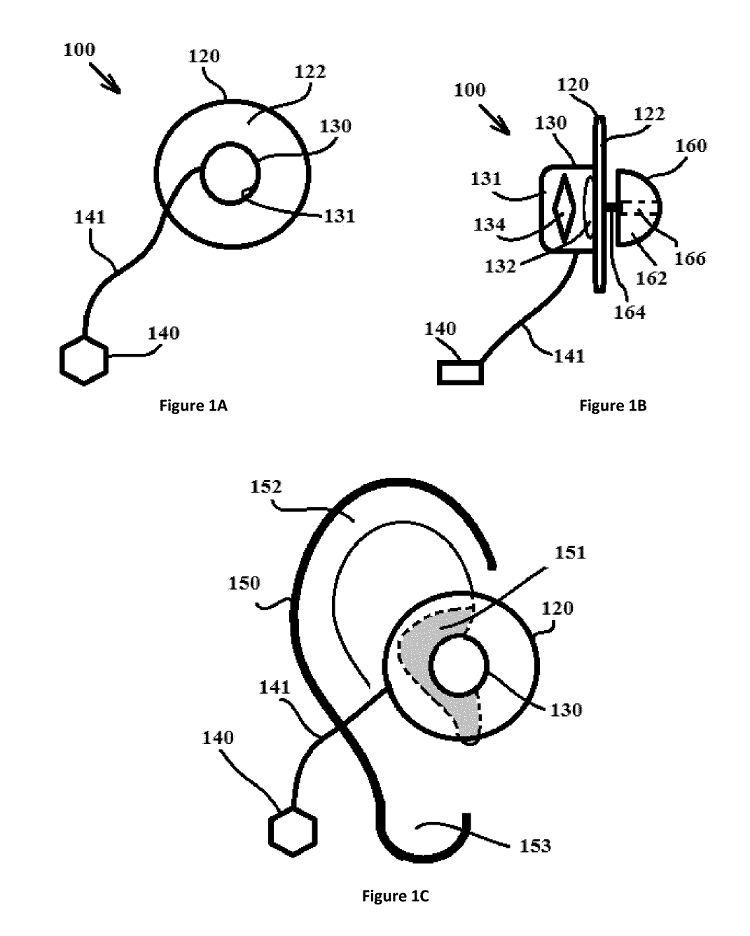

FIGS. 1A and 1B diagrammatically illustrate a device 100 according to exemplary embodiments of the present disclosure, and FIG. 1C diagrammatically illustrates a device 100 according to exemplary embodiments of the present disclosure with respect to a human ear 150, whose well known anatomy includes cavity 151, helix 152, and lobule 153.

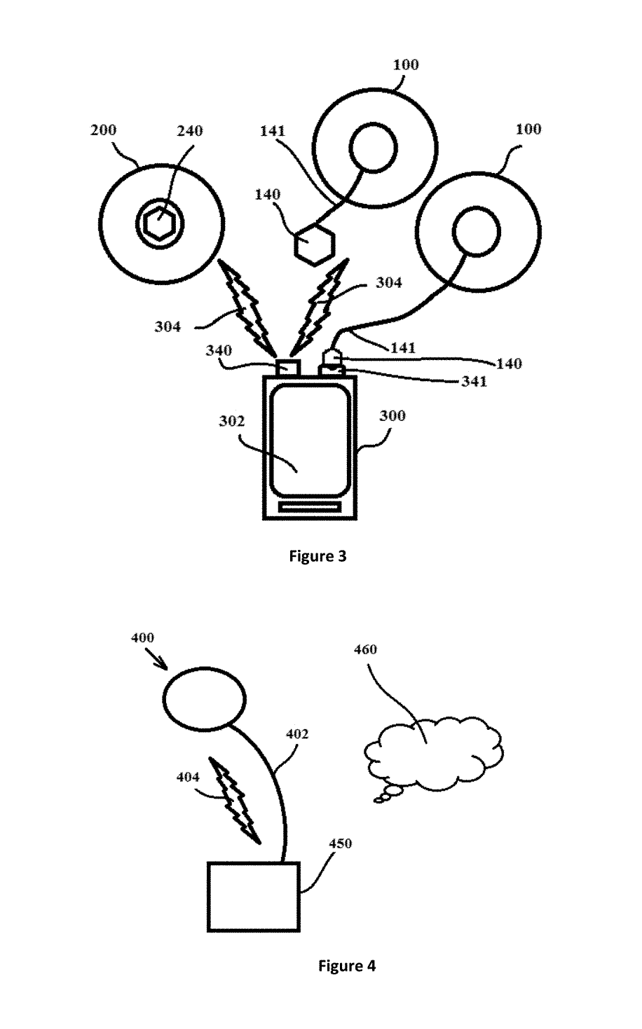

Referring to an example of FIGS. 1A (top view), 1B (side view), and 1C exemplary implementations of embodiments of the present disclosure provide an in-ear or on-ear device 100 that includes a sensing section 120 with at least one sensor 122 configured with respect to an ear bud or a headphone listening section 130 comprising housing 131 having disposed therein a speaker 132 and associated electronics 134 for outputting audio, such as without limitation speech and/or music, received from a connected device (for example as illustrated and described below with reference to FIG. 3) via connection section 140 comprising at least one of wired (for example, a conventional prong, or micro-USB, or other) or wireless (for example, a nearfield wireless transceiver) connection(s) external to housing 131 in communication with electronics 134 by a wired connection 141. Electronics 134 can include for example a microprocessor and a non-transient computer-readable memory, for receiving, processing and/or storing: data associated with information sensed by sensing section 120; and/or information and/or commands from an external control/communication device (such as device 300 of FIG. 3 described below), and outputting data indicative of the sensed information to a connected device (such as device 300) for example via connection section 140.

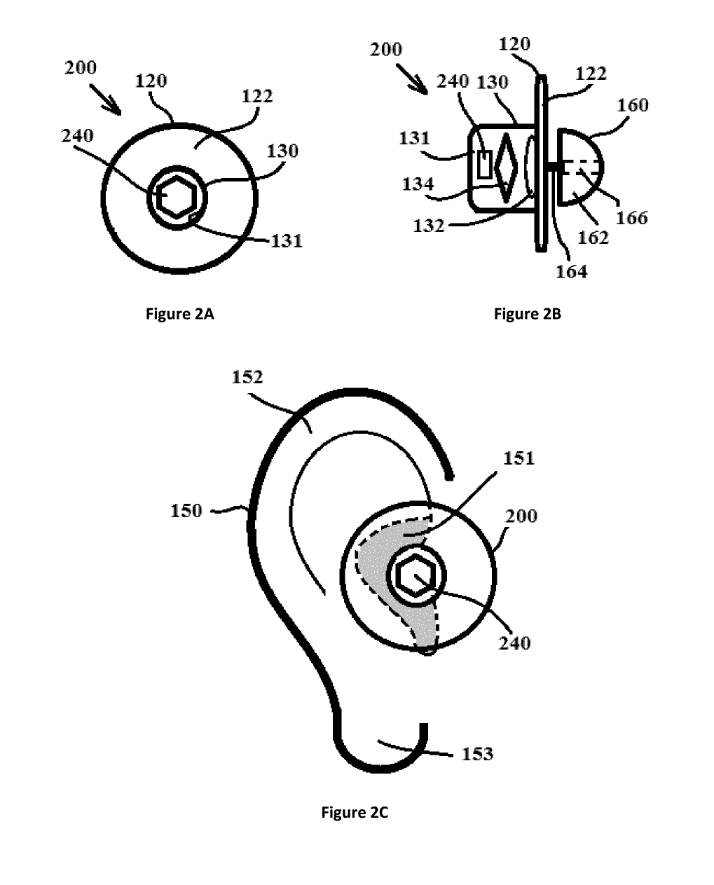

In an exemplary implementation, sensing section 120 is configured proximate to, or integral with, housing 131, for example as a resilient or flexible disc-like structure having one or more sensors 122 disposed thereon. In yet further exemplary implementation, device 100 (and device 200) further includes an insert 160 that can be attached to, or formed integral with, section 120 and/or section 130, and comprises a bud 162, for example removably attached to a stem 164 and made of flexible hypoallergenic material for secure and comfortable fitting inside cavity 151 with an open channel 166 to facilitate hearing of audio output from speaker 132. Insert 160 is configured for placement inside cavity 151 of ear 150, as diagrammatically illustrate in FIG. 1C, and also with respect to section 130 to facilitate hearing of audio output from speaker 132, and with respect to section 120 to facilitate accurate sensing by sensor 122 when placed inside cavity 151 (see FIGS. 1B, 1C, 2B, and 2C).

Referring to an example of FIGS. 2A (top view), 2B (side view), and 2C, other exemplary implementations of the embodiments of the present disclosure provide an in-ear or on-ear device 200 configured for wireless communication that includes a wireless transceiver 240, for example capable of nearfield wireless communication, disposed inside housing 131. According to an example of FIGS. 2A, 2B and 2C, built-in wireless transceiver 240 can be disposed within housing 131 and connected to electronics 134 and/or sensor 122) to wirelessly: (a) output data indicative of the information sensed by section 120 to a connected device; (b) receive audio, such as without limitation speech and/or music, from a connected device for output by speaker 132; and/or (c) receive command information, for example to control operation of electronics 134 and associated sensors 122, from a connected device.

Power to various components, such as electronics 134, sensor 122 and/or I/O device(s) 140, 240 can be provided by an internal power source (for example, disposed within housing 131), or an external power source (for example disposed within connection section 140, or a power source of a connected device (for example, when in wired connection with device 100, 200). Any of the aforementioned power sources can include, for example, a battery (disposable or rechargeable).

According to exemplary embodiments of the present disclosure, in-ear or on-ear device can connect to and exchange information with a connected device, such as a personal display device, a smartphone, and/or a desktop or any other computing and/or date storage device as illustrated in a diagrams of FIGS. 3 and 4. For example, connected device 300 including an interactive graphical user interface 302 can connect to, and communicate with, device 100 via a wired connection 141, for example by means of a wired connection 341 such as a USB port or the like. On the other hand, device 300 can selectively connect to, and communication with, one or more device(s) 200 and/or 100 via wireless connection 304, by means of a wireless communication transceiver 340, such as near field wireless communication transceiver. FIG. 4 is a block diagram illustrating an exemplary embodiment of the present disclosure providing a system 400, including measuring device 100 and/or device 200 in wired 402 and/or wireless (e.g., intra- or internet based) 404 communication with: external work station 450, which can provide and receive control information to/from device 100 and/or device 200, perform data processing and/or display and/or storage; and/or external data storage 460, which could be cloud-based, shared and/or secure. In the example of FIG. 4, sensed data collected and stored in an internal memory, which could be part of electronics 134, of device 100 and/or 200 can be downloaded for storage and/or processing via workstation 450 (which can also be a physical and/or a virtual server).

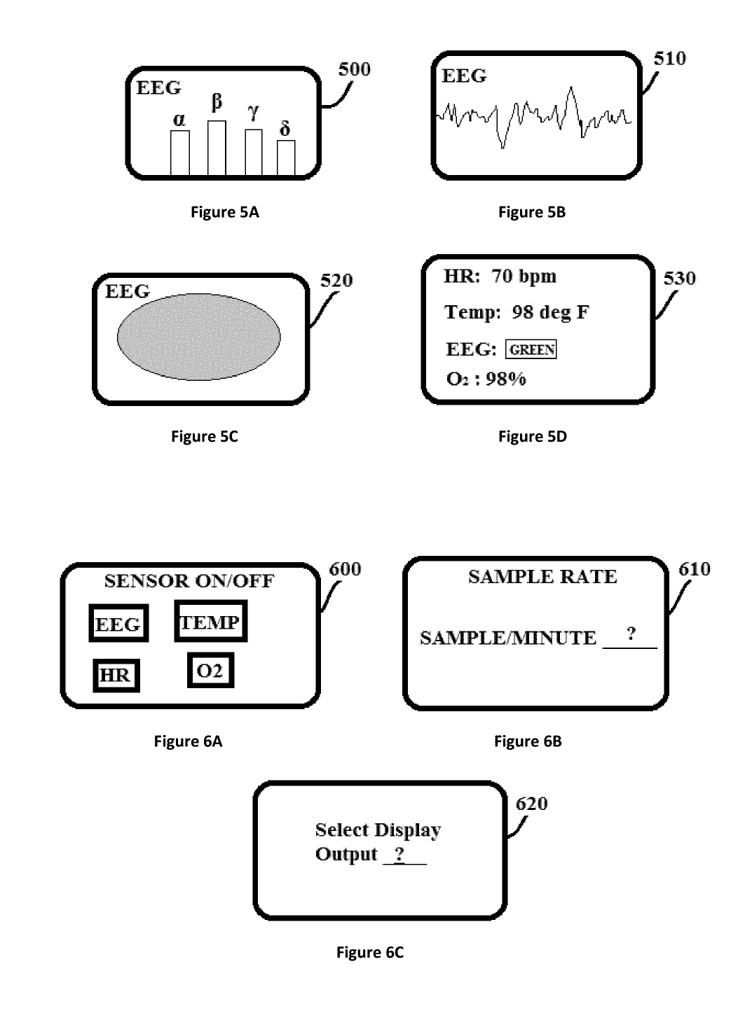

FIGS. 5A, 5B, 5C, and 5D diagrammatically illustrate examples of displays that can be generated on connected devices receiving information indicative of data sensed by sensing devices according to embodiments of the present disclosure. Any of the displays illustrated herein can be output on one or more display monitors individually and/or in combination with any and all other displays. FIG. 5A illustrates a display 500 (in an example of a bar graph format) of alpha (α), beta (β), gamma (γ), and delta (δ) waves' magnitude from sensed EEG data. FIG. 5B illustrates a display 510 of sensed EEG data output in a wave format—temporal frequency of the signals detected by EEG sensors indicative of brain activity in real time can range from less than 1 Hz to over 100 Hz. FIG. 5C illustrates display 520 of EEG data as a combined color output to provide a simple visual feedback of EEG in a color spectrum (for example: green implies “calm,” and “red” implies excited state). FIG. 5D illustrates display 530 were output from a plurality of sensors employed in device according to exemplary embodiments of the present disclosure (as described further below) can be provided selectively and/or simultaneously on one display, including for example: heart rate (HR), body temperature (Temp), EEG as a color pallet, and/or oxygen level (O₂).

FIGS. 6A, 6B, and 6C diagrammatically illustrate examples of interactive displays that can be generated on connected devices to provide a user interface (such as a graphical user interface) to facilitate control of an output generated by, and/or received from, any and all of the sensors of a device according to exemplary embodiments of the present disclosure. FIG. 600 illustrates an interactive display where one or more of the available outputs from sensor(s) provided in a device according to exemplary embodiments of the present disclosure can be selectively activated or deactivated. FIG. 6B illustrates an interactive display where operation and/or output (such as sampling rate) of one or more sensors can be selectively controlled and/or commanded. FIG. 6C illustrates an interactive display where the type of output (as illustrated for example in FIGS. 5A, 5B, 5C, or 5D) generated based on data received from one or more sensors of a device according to exemplary embodiments of the present disclosure can be selectively controlled. One of ordinary skill in the art would readily appreciate that a conventional control of volume output from an audio speaker of a device according to exemplary embodiment of the present disclosure can also be provided.

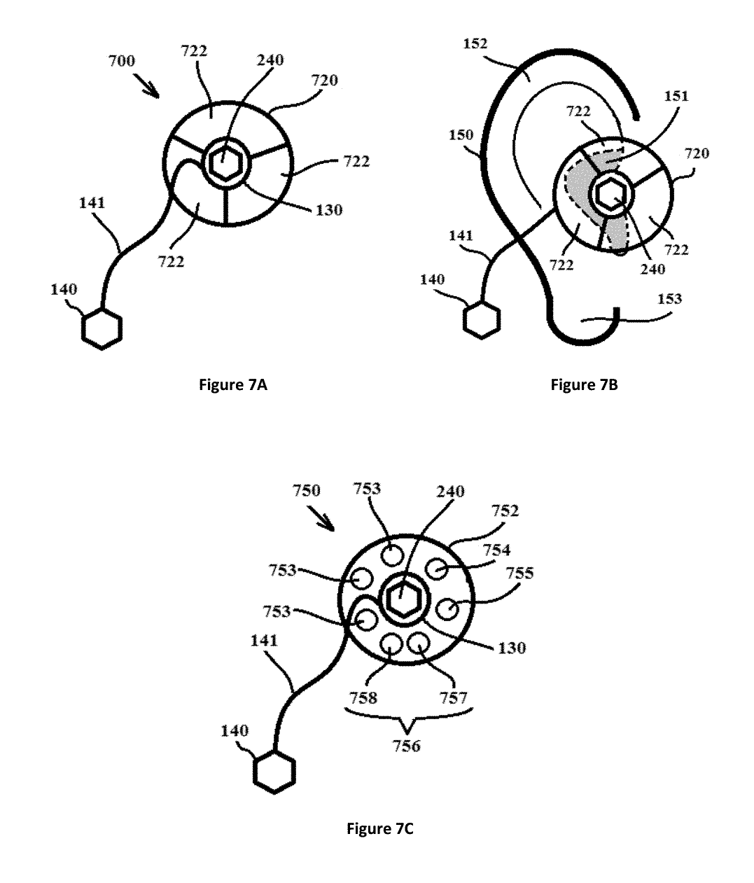

Referring to FIGS. 7A and 7B, exemplary implementations of the embodiments of the present disclosure provide an in-ear or on-ear device 700 that comprises a sensing section 720 with multiple EEG sensors 722 (for example, comprising metal plates in a disc-like arrangement) configured with respect to an ear bud or a headphone listening section 130 (see FIGS. 1A, 1B, 2A and 2B). According to an exemplary implementation, device 700 comprises: (a) an external connection section 140, including a wired connection 141, as described above with reference to the example of FIGS. 1A and 1B; and/or (b) a built-in wireless transceiver 240, as described above with reference to the example of FIGS. 2A and 2B. Also, electronics 134 can include for example a microprocessor and a non-transient computer-readable memory, for receiving, processing and/or storing: data associated with information sensed by each of the sensors 722 of sensing section 720; and/or information and/or commands from an external control and/or communication device (such as device 300 of FIG. 3 described above), and outputting data indicative of the sensed information to a connected device for example via connection section 140. In an exemplary implementation, device 700 further includes an insert 160 (see FIGS. 1B and 2B) configured for placement inside cavity 151 of ear 150, as diagrammatically illustrate in FIG. 7B, and also with respect to section 130 to facilitate hearing of audio output from speaker 132, and with respect to section 720 to facilitate accurate sensing by sensor 722 when placed inside cavity 151 (see FIGS. 1B, 1C, 2B, and 2C).

Referring to FIG. 7C, exemplary embodiments of the present disclosure provide an in-ear or on-ear device 750 configured similar to device 700, but comprising a sensing section 752 with multiple sensors configured with respect to an ear bud or a headphone listening section 130, for example on a disc-like structure. The sensors can include, without limitation, any one or all of: EEG sensor(s) 753 (for example, comprising magnet(s) and/or metal plate(s)); a pulse wave sensor 754 (for example, comprising a microphone); a temperature sensor 755; and an 02 sensor 756 (for example comprising a light source 757, such as an LED, and a corresponding photosensitive element 758 arranged with respect to light source 757).

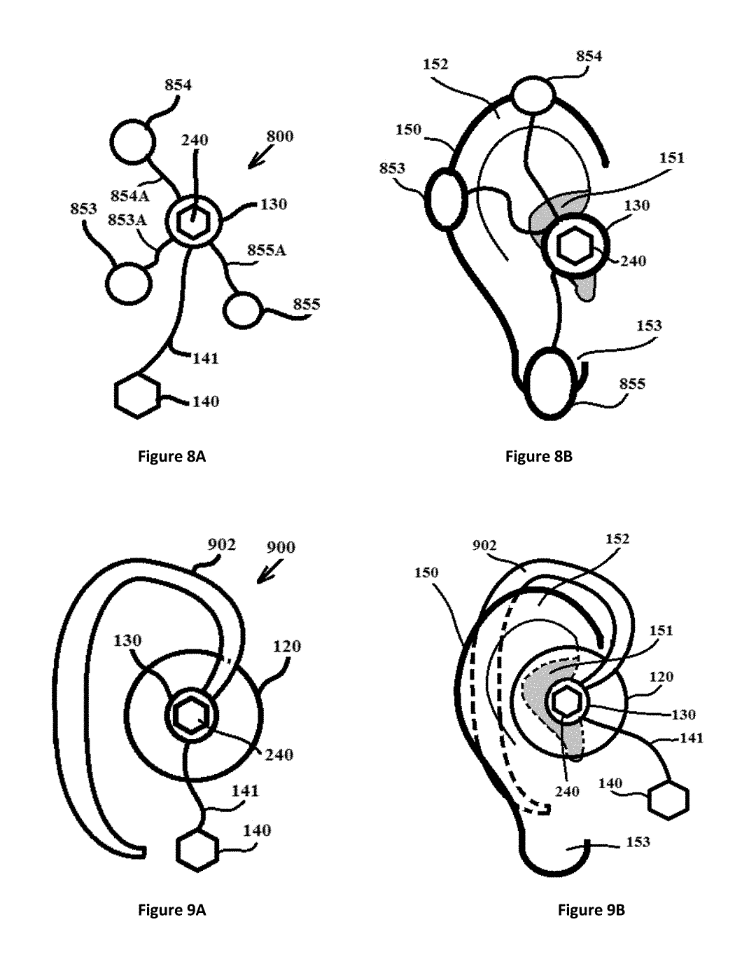

Referring to FIGS. 8A and 8B, exemplary implementations of embodiments of the present disclosure provide an in-ear or on-ear device 800 that comprises one or more sensors 853, 854, 855 configured remotely with respect to an ear bud or a headphone listening section 130 (see FIGS. 1A, 1B, 2A and 2B) for independent attachment to a portion of an ear 150. In an exemplary implementation, device 800 further includes an insert 160 (see FIGS. 1B and 2B) configured for placement inside cavity 151 of ear 150, as diagrammatically illustrate in FIG. 8B, and also with respect to section 130 to facilitate hearing of audio output from speaker 132 (see FIGS. 1B, 1C, 2B, and 2C). In an exemplary implementation, sensors 853, 854, 855 can comprise electrodes of an EEG sensor attached to helix 152 (for example as an ear cuff) and/or lobule 153 (for example as an earring if lobule is pierced, or a clip-on) when insert 160 of a device 800 is placed into cavity 151 of ear 150. In another exemplary implementation, any one of sensors 853, 854, 855 can be implemented as an individual sensor such as EEG sensor(s) 753, pulse wave sensor 754, temperature sensor 755, or O₂ sensor 756. In yet further exemplary implementation, connections 853A, 854A, 855A of respective sensors 853, 854, 855 to electronic 134 of section 130 can be implemented as decorative wired connections.

According to an exemplary implementation, device 800 comprises: (a) an external connection section 140, including a wired connection 141, as described above with reference to the example of FIGS. 1A and 1B; and/or (b) a built-in wireless transceiver 240, as described above with reference to the example of FIGS. 2A and 2B. Also, electronics 134 can include for example a microprocessor and a non-transient computer-readable memory, for receiving, processing and/or storing: data associated with information sensed by each of the sensors 853, 854, 855; and/or information and/or commands from an external control and/or communication device (such as device 300 of FIG. 3 described above), and outputting data indicative of the sensed information to a connected device for example via connection section 140.

An exemplary embodiment of the disclosure provides a device 900, which can be implemented as any one of the devices 100, 200, 700, 750, or 800 with a holder 902 attached to, or formed integral with, section 130 thereof, as illustrated in the example of FIG. 9A. Holder 902 can be configure in addition to an insert 160, or allow for omission of an insert 160, for placement behind helix 152 to facilitate securing section 130 of device 900 with respect to cavity 151, as illustrated in the example of FIG. 9B.

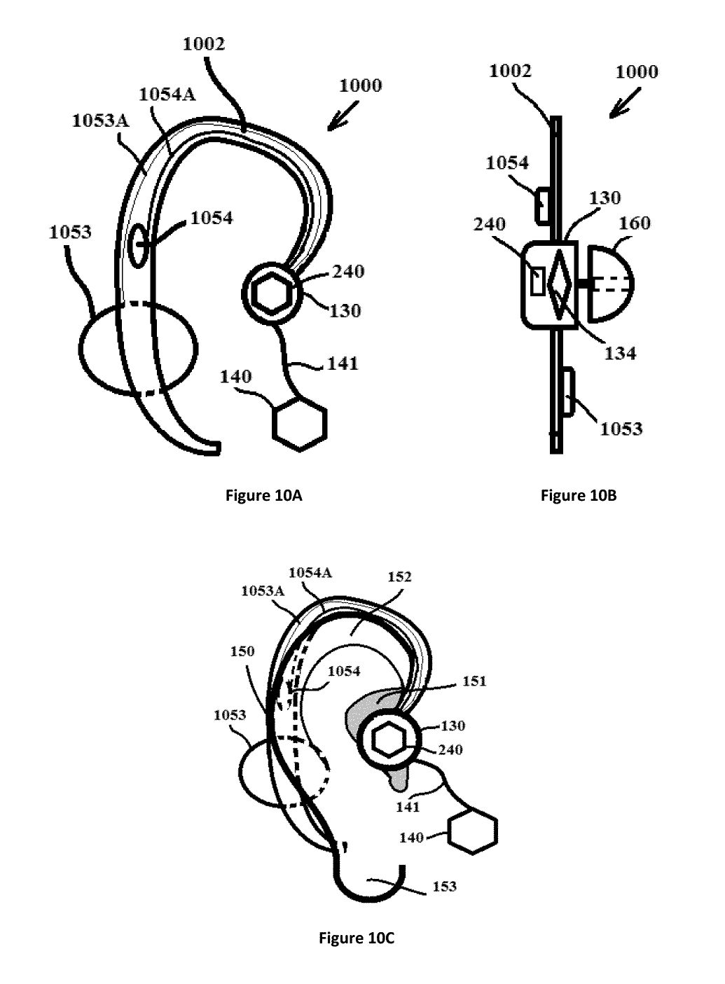

Yet another exemplary embodiment of the present disclosure provides a device 1000, which can be implemented as any one of the devices 100, 200, 700, 750, or 800 with a holder 1002, such that one or more sensors 1053, 1054 can be disposed on the holder 1002, as illustrated in FIGS. 10A, 10B, and 10C. In an exemplary implementation, sensors 1053, 1054 can comprise electrodes of an EEG sensor for example placed in contact with body behind the ear, such as sensor 1053, or in contact with the ear, such as sensor 1054. In another exemplary implementation, any one of sensors 1053, 1054 can be implemented as an individual sensor such as EEG sensor(s) 753, pulse wave sensor 754, temperature sensor 755, or 02 sensor 756. According to an exemplary implementation holder 1002 can be configured to accommodate internally, or externally, one or more wired connections 1053A, 1054A of respective sensors 1053, 1054 to electronics 134 for communication of sensed data.

According to an exemplary implementation, device 1000 comprises: (a) an external connection section 140, including a wired connection 141, as described above with reference to the example of FIGS. 1A and 1B; and/or (b) a built-in wireless transceiver 240, as described above with reference to the example of FIGS. 2A and 2B. Also, electronics 134 can include for example a microprocessor and a non-transient computer-readable memory, for receiving, processing and/or storing: data associated with information sensed by each of the sensors 1053, 1054; and/or information and/or commands from an external control and/or communication device (such as device 300 of FIG. 3 described above), and outputting data indicative of the sensed information to a connected device for example via connection section 140.

In exemplary implementations of the embodiments of the present disclosure, any one of the devices 100, 200, 700, 750, 800, 900, and 1000 can be configured with respect to one or both ears as shown in FIGS. 11A and 11B, such that any one of, or all of, the sensors can be configure with respect to one ear or both ears. In an exemplary implementation including a configuration where sensors are provided with respect to a listening section of each ear, only one microprocessor and only one transceiver (wired and/or wireless) may be required within one of the listening sections, such that information sensed by any one or all of the sensors disposed with respect to each ear can be received and processed by the microprocessor. In an exemplary implementation, EEG sensor can comprise two or more electrodes in contact with each ear. In yet further exemplary implementation as illustrated diagrammatically in FIG. 11B, where headphones comprise on-ear configuration including headphone 1100, an insert 160 may not be required since sensor section 120 can be disposed in the headphone 1100 such that the sensors make sufficient contact with the body, for example the ear, due to tension provided by headpieces 1102 (such as in a conventional headset configuration).

In an exemplary implementation of the embodiments of the present disclosure, a microphone can be configured with, or replace, speaker 132 inside housing 131, for sensing a pulse wave indicative of a heart rate. In yet another exemplary implementation, 02 sensor can measure arterial pulse. In still further exemplary implementation, pulse and/or heart rate data sensed by any one of the sensors of devices 100, 200, 700, 750, 800, 900, and 1000 can be coupled with data sensed by other on-body sensors, such as a pulse sensor disposed in a watch, a wristband, or an ankle band.

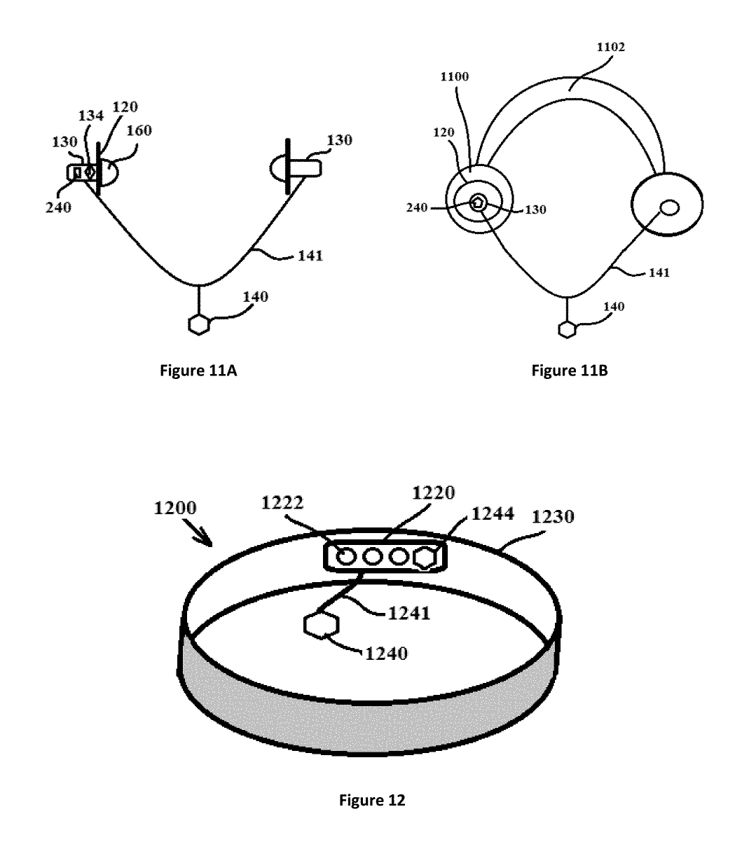

In yet another exemplary implementation, referring to FIG. 12, another on-body sensor 1200 can be provided in the form of a headband 1230 comprising sensing section 1220 including any one or more sensors 1222 as described above in wired or wireless communication, for example with any one of devices 100, 200, 700, 750, 800, 900, and 1000, and/or an external connected device, such as device 300, by means of an internal wireless transceiver 1244 (such as transceiver 240 described above) and/or an external wired or wireless connection 1241, 1240 (such as connection 140, 141 described above).

While the present disclosure has been shown and described with reference to certain exemplary embodiments thereof, it will be understood by those skilled in the art that various changes in form and details may be made therein without departing from the spirit and scope of the present disclosure.

Other objects, advantages and salient features of the disclosure will become apparent to those skilled in the art from the details provided, which, taken in conjunction with the annexed drawing figures, disclose exemplary embodiments of the disclosure.

Those of skill in the art further appreciate that the various illustrative logical blocks, modules, circuits, and algorithm steps described in connection with the embodiments disclosed herein may be implemented as electronic hardware, computer software, or combinations of both. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

The above-presented description and figures are intended by way of example only and are not intended to limit the illustrative embodiments in any way except as set forth in the appended claims. It is particularly noted that various technical aspects of the various elements of the various exemplary embodiments that have been described above can be combined in numerous other ways, all of which are considered to be within the scope of the disclosure.

Accordingly, although exemplary embodiments have been disclosed for illustrative purposes, those skilled in the art will appreciate that various modifications, additions, and substitutions are possible. Therefore, the disclosure is not limited to the above-described embodiments, but may be modified within the scope of appended claims, along with their full scope of equivalents.

I claim:

1. A health data monitoring device for comprising:
a listening section including a speaker outputting an audio signal received from an external source; and
a sensor section configured in conjunction with said listening section and including at least one sensor,
wherein
when said listening section is positioned with respect to an ear of a person to enable listening of said audio signal, said at least one sensor is configure for sensing health data associate with said person.

2. The device of claim 1, wherein said at least one sensor comprises an electroencephalogram (EEG) sensor.

3. The device of claim 1, wherein said listening section comprises an ear bud.

4. The device of claim 1, wherein said listening section comprises a plurality of sensors including at least one of an EEG sensor, a pulse wave sensor, a heart rate sensor, a temperature sensor, and an oxygen sensor, and
when said listening section is positioned with respect to an ear of a person to enable listening of said audio signal, said at least one of said plurality of sensors is configure for sensing health data associate with said person.

5. The device of claim 4, wherein
when said listening section is positioned with respect to an ear of a person to enable listening of said audio signal, said plurality of sensors are configure for sensing health data associate with said person

6. The device of claim 1, further comprising
a microprocessor and a transceiver configure to receive and process said sensed health data, and to transmit information indicative of said processed health data to an external device for at least one of further processing and visual and/or audio display.

7. The device of claim 6, wherein
said transceiver is configure for at least one of wired and wireless communication.

8. The device of claim 6, further comprising
a non-transient computer-readable memory for recording and/or storing at least a portion of said information indicative of said sensed health data.

9. The device of claim 8, wherein
at least one of said microprocessor, transceiver, and memory is configured within a housing accommodating said listening section.

10. The device of claim 6, wherein
said microprocessor and said transceiver are configured to receive at least one of command and data input from an external device via a wired or wireless communication; and
said microprocessor controls operation of said at least one sensor according to at least one of said received command and data input.

11. A health data monitoring system comprising:
the first health data monitoring device as claimed in any one of claims 1 to 10; and
an external device in wired or wireless communication with said health data monitoring device,
wherein said external device
transmits to said first health data monitoring device at least one of said audio signal and a control signal for controlling operation of said first health data monitoring device,
receives data indicative of said health data sensed by said at least one sensor, and
outputs at least one of visual and audio representation of said sensed health data.

12. The system comprising:
a first health data monitoring device comprising:
a listening section including a speaker outputting an audio signal received from an external source; and
a sensor section configured in conjunction with said listening section and including at least one sensor,
wherein
when said listening section is positioned with respect to an ear of a person to enable listening of said audio signal, said at least one sensor is configure for sensing health data associate with said person;
a second health data monitoring in wired or wireless communication with at least one of said first health data monitoring device and said external device,
wherein said first and second health data monitoring devices are configure to output said audio signal from said external device in a stereophonic format,
said output of said at least one sensor of said first health data monitoring device is processed in coordination with said output of said at least one sensor of said second health data monitoring device, and
said external device transmits to at least one of said first and second health data monitoring devices at least one of said audio signal and a control signal for controlling operation of said at least one of first and second health data monitoring devices.

13. The system of claim 12, wherein
said at least one sensor comprises an electroencephalogram (EEG) sensor.

14. The system of claim 12, wherein
said listening section comprises a plurality of sensors including at least one of an EEG sensor, a pulse wave sensor, a heart rate sensor, a temperature sensor, and an oxygen sensor, and
when said listening section is positioned with respect to an ear of a person to enable listening of said audio signal, said at least one of said plurality of sensors is configure for sensing health data associate with said person.

15. A method of monitoring health data, the method comprising:
positioning at least one the health data monitoring device with respect to an ear of a person, said at least one health data monitoring device comprising:
a listening section including a speaker outputting an audio signal received from an external source, and
a sensor section configured in conjunction with said listening section and including at least one sensor;
enable listening of an audio signal output by said listening section;
sensing health data associate with said person by said at least one sensor of said health data monitoring device; and
outputting said sensed health data.

16. The method of claim 15, wherein said at least one sensor comprises an electroencephalogram (EEG) sensor.

17. The method of claim 15, wherein
said listening section comprises a plurality of sensors including at least one of an EEG sensor, a pulse wave sensor, a heart rate sensor, a temperature sensor, and an oxygen sensor, and
when said listening section is positioned with respect to an ear of a person to enable listening of said audio signal, said at least one of said plurality of sensors is configure for sensing health data associate with said person.

18. The method of claim 15, further comprising receiving said sensed health data and outputting information indicative of said sensed health data in a visual and/or audible format.

19. The method of claim 15, further comprising controlling at least one of said audio signal, operation of said sensing section, and said visual and/or audible format of said output, by an external controller in wired or wireless communication with said health data monitoring device.
